(19) United States
(12) Patent Application Publication
(10) Pub. No.: US 2011/0184061 A1
(43) Pub. Date: Jul. 28, 2011

(54) THIOCOLCHICINE AND COLCHICINE ANALOGS, METHODS OF MAKING AND METHODS OF USE THEREOF

(76) Inventors: Kurt R. Nielsen, Chadds Ford, PA (US); Rajesh Manchanda, Sudbury, MA (US); Tong Sun, Marlton, NJ (US); Shawn G. Watson, Cherry Hill, NJ (US)

(21) Appl. No.: 13/010,266

(22) Filed: Jan. 20, 2011

Related U.S. Application Data
(60) Provisional application No. 61/297,316, filed on Jan. 22, 2010.

(51) Int. Cl. A61K 31/265; C07C 69/96; A61P 29/00; A61P 21/02; A61P 35/00

(52) U.S. Cl. 514/512; 558/271

(57) ABSTRACT

Disclosed herein are thiocolchicine and colchicine analogs and derivatives suitable for use as a muscle relaxant, an anti-inflammatory agent, an anti-gout agent, an anti-proliferative agent, or an anti-cancer agent; methods of making the compounds, and compositions comprising the compounds.

### CROSS REFERENCE TO RELATED APPLICATION

This application claims the benefit of U.S. Provisional Application Ser. No. 61/297,316 filed Jan. 22, 2010, which is hereby incorporated by reference in its entirety.

### BACKGROUND

Thiocolchicine and colchicine are known semisynthetic and natural alkaloids, respectively. Thiocolchicine is an inhibitor of microtubules by specific binding to tubulin. Colchicine is a known gout suppressant and agent for the treatment of Familial Mediterranean Fever.

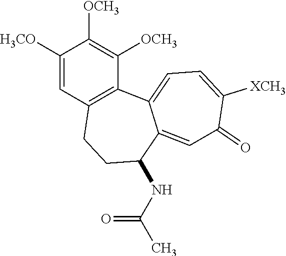

X═O, colchicine; X═S, thiocolchicine

The thiocolchicine derivative thiocolchicoside (N-[(7S)-3-(β-D-glucopyranosyloxy)-5,6,7,9-tetrahydro-1,2-dimethoxy-10-(methylthio)-9-oxobenzo[a]heptalen-7-yl]-acetamide also known as 3-demethylthiocolchicine glucoside; CAS Registry No. 602-41-5) is a known skeletal muscle relaxant. Studies have suggested that thiocolchicoside is metabolized in vivo into an aglycone derivative via deglycosylation and subsequent formation of a 3-O-glucuronidated aglycone derivative. See, Trellu et al., “New metabolic and pharmacokinetic characteristics of thiocolchicoside and its active metabolite in healthy humans”, Fundamental & Clinical Pharmacology, 18, (2004) 493-501. The aglycone derivative exhibited no muscle relaxant activity in a rat model while the 3-O-glucuronidated aglycone derivative was found to exhibit muscle relaxant activity similar to that of thiocolchicoside. Id.

There remains a need in the art for new compounds exhibiting muscle relaxant activity, anti-gout activity, or other therapeutic benefits having greater safety profile, activity, or therapeutic index than thiocolchicoside, thio colchicine, or colchicine.

### SUMMARY

In one embodiment, a compound according to structure (I)

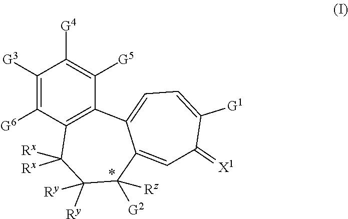

wherein

A) when G¹ is SeR¹, TeR¹, PoR¹, P(R²)₂, N(R³)₂, or Si(R⁴)₃;
R¹ is hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl, wherein when R¹ is not hydrogen it is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R² is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl; or the two R² form a 5- or 6-membered cyclic group having 0, 1, or 2 additional heteroatoms chosen from N, O, or S; wherein each occurrence of R² that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R³ is independently chosen from C₈-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R³ form a 5- or 6-membered cyclic group having 0, 1, or 2 additional heteroatoms chosen from N, O, or S; wherein each occurrence of R³ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
wherein each occurrence of R⁴ independently is hydrogen, C₁-C₁₀ alkyl, C₂-C₁₀ alkenyl, or C₃-C₇ cycloalkyl; wherein each occurrence of R⁴ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;

X¹ is O or S;

G² is —OH, —OR^{a}, —SR^{a}, —N(R^{b})₂, heterocycloalkyl, aryl, heteroaryl, or a carbohydrate group linked via an O-, N- or S-glycosidic bond;
wherein R^{a} is C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, epoxide, carbonate, or N-succinimidyl group; wherein R^{a} is optionally substituted with 1, 2, or 3 substituents independently chosen from hydroxy, amino, cyano, halogen, C₁-C₆alkyl, C_{z}—C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
wherein each occurrence of R^{b} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C_{z}—C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{b} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{b} form a 5- or 6-membered cyclic group having 0, 1, or 2 additional heteroatoms chosen from N, O, or S;

each occurrence of R^{x}, R^{y}, and R^{z} is independently chosen from hydrogen, halogen, C₁-C₃alkyl, or C₁-C₃haloalkyl, or
where the two R^{x} groups, the two R^{y} groups, one R^{x} and one R^{y} group, or one R^{y} and R^{z} group form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;

G³ is —OH, —OR^{a}, —N(R^{c})₂, heterocycloalkyl, aryl, heteroaryl, or a carbohydrate group linked via an O-, N- or S-glycosidic bond;
wherein each occurrence of R^{c} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{c} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{c} form a 5- or 6-membered cyclic group having 0, 1, or 2 additional heteroatoms chosen from N, O, or S;

G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;

G⁵ is —OH, —OR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁶ is H, —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined; or where the G³ and G⁴ groups or G⁴ and G⁵ groups or G³ and G⁶ groups form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;

B) wherein when at least one of G², G³, G⁴, G⁵ or G⁶ is —OR^{a}, —SR^{a}, —N(R^{b})₂, or —N(R^{c})₂ wherein a R^{a}, a R^{b} or a R^{c} is -L-PEG, then the remaining G², G³, G⁴, G⁵ and G⁶ are as previously defined;

G¹ is OR¹, SR¹, SeR¹, TeR¹, PoR¹, P(R²)₂, N(R⁵)₂, or Si(R⁴)₃, where R¹, R² and R⁴ are as previously defined;
wherein each occurrence of R⁵ is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R⁵ form a 5- or 6-membered cyclic group having 0, 1, or 2 additional heteroatoms chosen from N, O, or S; wherein each occurrence of R⁵ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;

X¹ is O or S; and

R^{x}, R^{y}, and R^{z} are as previously defined;

C) wherein when at least one of R^{x}, R^{y}, and R^{z} is not hydrogen, then the remaining R^{x}, R^{y}, and R^{z} are as previously defined;

G¹ is OR¹, SR¹, SeR¹, TeR¹, PoR¹, P(R²)₂, N(R⁵)₂, or Si(R⁴)₃, where R¹, R², R⁴ and R⁵ are as previously defined;

X¹ is O or S;

G² is —OH, —OR^{a}, —SR^{a}, —N(R^{b})₂ heterocycloalkyl, aryl, heteroaryl, or a carbohydrate group linked via an O-, N- or S-glycosidic bond, wherein R^{a} and R^{b} are as previously defined;

G³ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;

G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined; and

G⁵ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;

G⁶ is H, —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined; or

D) wherein

G¹ is OR¹ or SR¹;
R¹ is hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl, wherein when R¹ is not hydrogen it is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;

X¹ is O or S;

G² is —OH, —OR^{a}, —SR^{a}, —N(R^{b})₂, heterocycloalkyl, aryl, heteroaryl, or a carbohydrate group linked via an O-, N- or S-glycosidic bond;
wherein R^{a} is C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, epoxide, carbonate, or N-succinimidyl group; wherein R^{a} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
wherein each occurrence of R^{b} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{b} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{b} form a 5- or 6-membered cyclic group having 0, 1, or 2 additional heteroatoms chosen from N, O, or S;

each occurrence of R^{x}, R^{y}, and R^{z} is independently chosen from hydrogen, halogen, C₁-C₃alkyl, or C₁-C₃haloalkyl, or
where the two R^{x} groups, the two R^{y} groups, one R^{x} and one R^{y} group, or one R^{y} and R^{z} group form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;

G³ is —OH, —OR^{a}, —SR^{a}, —N(R^{c})₂, heterocycloalkyl, aryl, heteroaryl, or a carbohydrate group linked via an O-, N- or S-glycosidic bond;
wherein each occurrence of R^{c} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{c} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{c} form a 5- or 6-membered cyclic group having 0, 1, or 2 additional heteroatoms chosen from N, O, or S;

G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;

G⁵ is —OH, —OR^{a}, —SR^{a}, —N(R^{c})₂, heterocycloalkyl, aryl, heteroaryl, or a carbohydrate group linked via an O-, N- or S-glycosidic bond, and Wand R^{c} are as previously defined;

G⁶ is H, —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
or where the G³ and G⁴ groups or G⁴ and G⁵ groups or G³ and G⁶ groups form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; with the proviso that for D)

1) when G⁶ is H or OMe, G⁵ is OMe, X¹ is O, G¹ is OH, OMe or SMe, both R^{x} is H, both R^{y} is H, and R^{z} is H, then G³ and G⁴ do not form a 5-membered ring containing the group —OCH₂O— or —CH₂CH₂ CH₂—;

2) when G⁶ is H, G⁵ is OMe, X¹ is O, G¹ is OMe, both R^{x} is H, both R^{y} is H, and R^{z} is H, then G³ and G⁴ do not form a 6-membered ring containing the group —CH═CH—CH═CH—;

3) when G⁶ is H, G⁵ is OMe, X¹ is O, G¹ is SMe, both R^{x} is H, both R^{y} is H, and R^{z} is H, then G³ and G⁴ do not form a fused 6-membered ring containing two nitrogen atoms;

4) when G⁶ is H, G³ is OMe, X¹ is O, G¹ is SMe, both R^{x} is H, both R^{y} is H, and R^{z} is H, then G⁴ and G⁵ do not form a fused 6-membered ring containing two nitrogen atoms;

5) when G⁵ is OMe, G⁴ is OMe, X¹ is O, G¹ is SMe, both R^{x} is H, both R^{y} is H, and R^{z} is H, then G³ and G⁶ do not form a 5-membered ring containing the group —O—CH═N—; or

6) when G⁶ is H, G³ is OMe, X¹ is O, G¹ is OH or OMe, both R^{x} is H, both R^{y} is H, and R^{z} is H, then G⁴ and G⁵ do not form a 5-membered ring containing the group —OCH₂O—;

or a pharmaceutically acceptable salt, solvate, hydrate, crystalline form, non-crystalline form, or stereoisomer thereof.

In another embodiment, a compound according to structure (VII), (VIII), or (IX)

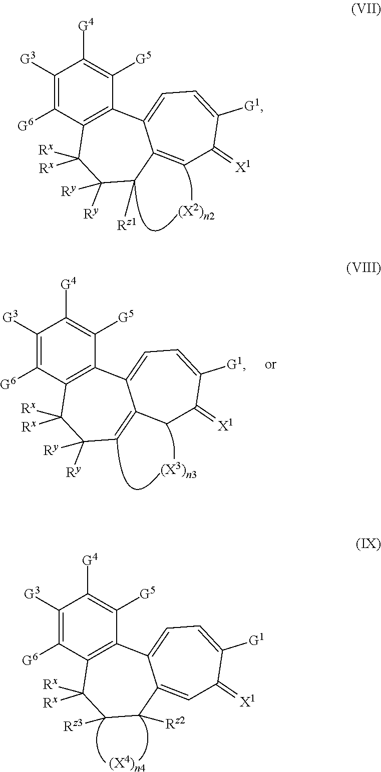

wherein G¹ is OR¹, SR¹, SeR¹, TeR¹, PoR¹, P(R²)₂, N(R⁵)₂, or Si(R⁴)₃, where
R¹ is hydrogen, C₁-C₁₀alkyl, C₂-C₁₀ alkenyl, C₃-C₇ cycloalkyl, C₁-C₁₀haloalkyl, heterocycloalkyl, aryl, or heteroaryl, wherein when R¹ is not hydrogen it is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R² is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl; or the two R² form a 5- or 6-membered cyclic group having 0, 1, or 2 additional heteroatoms chosen from N, O, or S; wherein each occurrence of R² that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R⁴ independently is hydrogen, C₁-C₁₀ alkyl, C₂-C₁₀ alkenyl, or C₃-C₇ cycloalkyl; wherein each occurrence of R⁴ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R⁵ is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, hetero aryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R⁵ form a 5- or 6-membered cyclic group having 0, 1, or 2 additional heteroatoms chosen from N, O, or S; wherein each occurrence of R⁵ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;

X¹ is O or S;

G³ is —OH, —OR^{a}, —SR^{a}, —N(R^{c})₂, heterocycloalkyl, aryl, heteroaryl, or a carbohydrate group linked via an O-, N- or S-glycosidic bond;
wherein each occurrence of R^{c} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{c} is optionally substituted with 1, 2, or 3 substituents independently chosen from hydroxy, amino, cyano, halogen, C₁-C₆alkyl, C_{z}—C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{c} form a 5- or 6-membered cyclic group having 0, 1, or 2 additional heteroatoms chosen from N, O, or S;

G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;

G⁵ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;

G⁶ is H, —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
or where the G³ and G⁴ groups or G⁴ and G⁵ groups or G³ and G⁶ groups form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;

each occurrence of R^{x}, and R^{y} is independently chosen from hydrogen, halogen, C₁-C₃alkyl, or C₁-C₃haloalkyl

each occurrence of X² is independently O, N, NR^{b}, CR^{b}, or CR^{b}₂ to form a cyclic group, where R^{b} is wherein each occurrence of R^{b} is independently chosen from hydrogen, C₁-C₁₀ alkyl, C₂-C₁₀alkenyl, C₃-C₇ cycloalkyl, C₁-C₁₀ haloalkyl, heterocycloalkyl, aryl, heteroaryl, C₁-C₆ alkoxycarbonyl, C_{z}—C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{b} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{b} form a 5- or 6-membered cyclic group having 0, 1, or 2 additional heteroatoms chosen from N, O, or S;

n² is 2 or 3;

R^{z1} is hydrogen or a bond forming the ring structure containing X²;

each occurrence of X³ is O, N, NR^{b}, CR^{b}, or CR^{b}₂ to form a cyclic group where R^{b} is as previously defined;

n³ is 2 or 3;

each instance of X⁴ is O, N, NR^{b}C, CR^{b}, or CR^{b}₂, to form a saturated or unsaturated cyclic group optionally fused to a 5- or 6-membered cyclic or aryl group where R^{b} is as previously defined;

n⁴ is 3 or 4;

R^{z2} is hydrogen or a bond forming the ring structure containing X⁴; and

R^{z3} is hydrogen, hydroxyl, or a bond forming the ring structure containing X⁴;

with the proviso that

a. for structure (VII)
1) when G⁶ is H, G³ is OMe, G⁴ is OMe, G⁵ is OMe, X¹ is O, G¹ is SMe, both R^{x} are H, and both R^{y} are H, then n² is not 2, R^{z1} is not a bond, and X² is not part of a 5-membered ring containing the group ═N—O— (where the oxygen is attached to the tropone ring), or
2) when G⁶ is H, G³ is OMe, G⁴ is OMe, G⁵ is OMe, X¹ is O, G¹ is OMe, both R^{x} is H, and both R^{y} is H, then n² is not 2, R^{z1} is not H, and X² is not part of a 5-membered ring containing the group —N(Ac)—C(OH)(Me)— (where the carbon is attached to the tropone ring);

b. for structure (VIII)
when G⁶ is H, G³ is OMe, G⁴ is OMe, G⁵ is OMe, X¹ is O, G¹ is OMe, both R^{x} is H, and both R^{y} is H, then n³ is not 2 and X³ is not part of a 5-membered ring containing the group —NH—C(CHO)═ or —NH—C(Me)═ where the doublebond is bonded to the tropone ring; or

c. for structure (IX)
1) when G⁶ is H, G³ is OMe, G⁴ is OMe, G⁵ is OMe, X¹ is O, G¹ is OMe or SMe, and both R^{x} is H, then n⁴ is not 4 and X⁴ is not part of a fused 6-membered ring containing the group

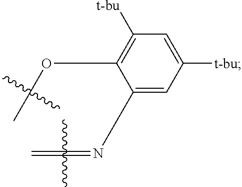

or
2) when G⁶ is H, G³ is OMe, G⁴ is OMe, G⁵ is OMe, X¹ is O, G¹ is OMe, and both R^{x} is H, then n⁴ is not 3 and X⁴ is not part of a 5-membered ring containing the group ═N—N(Ac)—CH═,
or a pharmaceutically acceptable salt, solvate, hydrate, crystalline form, non-crystalline form, or stereoisomer thereof.

Also disclosed are pharmaceutical compositions comprising a (thio)colchicinoid compound, as well as methods of treating a patient in need thereof by administration of a (thio)colchicinoid compound or a composition comprising a (thio)colchicinoid compound to the patient.

These and other embodiments, advantages and features of the present invention are provided in subsequent sections.

### DETAILED DESCRIPTION

Disclosed herein are thiocolchicine and colchicine analogs and derivatives having a (thio)colchicinoid three ring skeleton (“(thio)colchicinoid compound”), compositions comprising a (thio)colchicinoid compound, and uses thereof, particularly as a muscle relaxant, anti-gout agent, anti-proliferative agent, anti-cancer agent, or anti-inflammatory agent. Certain (thio)colchicinoid compounds are suitable for use as a muscle relaxant while at the same time being non-sedating.

In one embodiment, the (thio)colchicinoid compound comprises a compound according to structure (I)

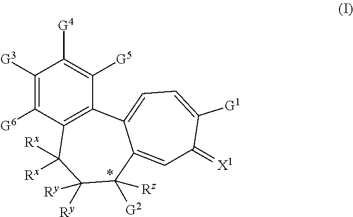

wherein A), B), C) or D) applies
A) wherein when G¹ is SeR¹, TeR¹, PoR¹, P(R²)₂, N(R³)₂, or Si(R⁴)₃;
   R¹ is hydrogen, C₁-C₁₀ alkyl, C₂-C₁₀ alkenyl, C₃-C₇ cycloalkyl, C₁-C₁₀haloalkyl, heterocycloalkyl, aryl, or heteroaryl, wherein when R¹ is not hydrogen it is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
   each occurrence of R² is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl; or the two R² form a 5- or 6-membered cyclic group having 0, 1, or 2 additional heteroatoms chosen from N, O, or S; wherein each occurrence of R² that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
   each occurrence of R³ is independently chosen from C₈-C₁₀ alkyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R³ form a 5- or 6-membered cyclic group having 0, 1, or 2 additional heteroatoms chosen from N, O, or S; wherein each occurrence of R³ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
   wherein each occurrence of R⁴ independently is hydrogen, C₁-C₁₀ alkyl, C₂-C₁₀ alkenyl, or C₃-C₇ cycloalkyl; wherein each occurrence of R⁴ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
   X¹ is O or S;
   G² is —OH, —OR^{a}, —SR^{a}, —N(R^{b})₂, heterocycloalkyl, aryl, heteroaryl, or a carbohydrate group linked via an O-, N- or S-glycosidic bond;
   wherein R^{a} is C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, epoxide, carbonate, or N-succinimidyl group; wherein R^{a} is optionally substituted with 1, 2, or 3 substituents independently chosen from hydroxy, amino, cyano, halogen, C₁-C₆alkyl, C_{z}—C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
   wherein each occurrence of R^{b} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C_{z}—C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{b} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{b} form a 5- or 6-membered cyclic group having 0, 1, or 2 additional heteroatoms chosen from N, O, or S;
   each occurrence of R^{x}, R^{y}, and R^{z} is independently chosen from hydrogen, halogen, specifically fluoro, C₁-C₃alkyl, or C₁-C₃haloalkyl, or
   where the two R^{x} groups, the two R^{y} groups, one R^{x} and one R^{y} group, or one R^{y} and R^{z} group form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;

G³ is —OH, —OR^{a}, —SR^{a}, —N(R^{c})₂, heterocycloalkyl, aryl, heteroaryl, or a carbohydrate group linked via an O-, N- or S-glycosidic bond;
wherein each occurrence of R^{c} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{c} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{c} form a 5- or 6-membered cyclic group having 0, 1, or 2 additional heteroatoms chosen from N, O, or S;
G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁵ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁶ is H, —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
or where the G³ and G⁴ groups or G⁴ and G⁵ groups or G³ and G⁶ groups form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
B) wherein when at least one of G², G³, G⁴, G⁵ or G⁶ is −—OR^{a}, —N(R^{b})₂, or —N(R^{c})₂ wherein a R^{a}, a R^{b} or a R^{c} is -L-PEG, then the remaining G², G³, G⁴, G⁵ and G⁶ are as previously defined;

G¹ is OR¹, SR¹, SeR¹, TeR¹, PoR¹, P(R²)₂, N(R⁵)₂, or Si(R⁴)₃, where R¹, R² and R⁴ are as previously defined;
wherein each occurrence of R⁵ is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R⁵ form a 5- or 6-membered cyclic group having 0, 1, or 2 additional heteroatoms chosen from N, O, or S; wherein each occurrence of R⁵ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄ alkyl)amino, —COOH, C₂-C₆alkoxycarbonyl, C₁-C₆haloalkyl, or C₁-C₆haloalkoxy;

X¹ is O or S; and
R^{x}, R^{y}, and R^{z} are as previously defined;
C) wherein when at least one of R^{x}, R^{y}, and R^{z} is not hydrogen, then the remaining R^{x}, R^{y}, and R^{z} are as previously defined;
G¹ is OR¹, SR¹, SeR¹, TeR¹, PoR¹, P(R²)₂, N(R⁵)₂, or Si(R⁴)₃, where R¹, R², R⁴ and R⁵ are as previously defined;
X¹ is O or S;
G² is —OH, —OR^{a}, —SR^{a}, —N(R^{b})₂, heterocycloalkyl, aryl, heteroaryl, or a carbohydrate group linked via an O-, N- or S-glycosidic bond, wherein R^{a} and R^{b} are as previously defined;
G³ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁵ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined; and
G⁶ is H, —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined; or
D) wherein when G¹ is OR¹ or SR¹;
R¹ is hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl, wherein when R¹ is not hydrogen it is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
X¹ is O or S;
G² is —OH, —OR^{a}, —SR^{a}, —N(R^{b})₂, heterocycloalkyl, aryl, heteroaryl, or a carbohydrate group linked via an O-, N- or S-glycosidic bond;
wherein R^{a} is C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, epoxide, carbonate, or N-succinimidyl group; wherein R^{a} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
wherein each occurrence of R^{b} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{b} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{b} form a 5- or 6-membered cyclic group having 0, 1, or 2 additional heteroatoms chosen from N, O, or S;
each occurrence of R^{x}, R^{y}, and R^{z} is independently chosen from hydrogen, halogen, specifically fluoro, C₁-C₃alkyl, or C₁-C₃haloalkyl, or
where the two R^{x} groups, the two R^{y} groups, one R^{x} and one R^{y} group, or one R^{y} and R^{z} group form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
G³ is —OH, —OR^{a}, —SR^{a}, —N(R^{c})₂, heterocycloalkyl, aryl, heteroaryl, or a carbohydrate group linked via an O-, N- or S-glycosidic bond;
wherein each occurrence of R^{c} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{c} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{c} form a 5- or 6-membered cyclic group having 0, 1, or 2 additional heteroatoms chosen from N, O, or S;
G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁵ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁶ is H, —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
or where the G³ and G⁴ groups or G⁴ and G⁵ groups or G³ and G⁶ groups form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
with the proviso for D) that
1) when G⁶ is H or OMe, G⁵ is OMe, X¹ is O, G¹ is OH, OMe or SMe, both R^{x} is H, both R^{y} is H, and R^{z} is H, then G³ and G⁴ do not form a 5-membered ring containing the group —OCH₂O— or —CH₂CH₂ CH₂—;
2) when G⁶ is H, G⁵ is OMe, X¹ is O, G¹ is OMe, both R^{x} is H, both R^{y} is H, and R^{z} is H, then G³ and G⁴ do not form a 6-membered ring containing the group —CH═CH—CH═CH—;
3) when G⁶ is H, G⁵ is OMe, X¹ is O, G¹ is SMe, both R^{x} is H, both R^{x} is H, and R^{z} is H, then G³ and G⁴ do not form a fused 6-membered ring containing two nitrogen atoms;
4) when G⁶ is H, G³ is OMe, X¹ is O, G¹ is SMe, both R^{x} is H, both R^{y} is H, and R^{z} is H, then G⁴ and G⁵ do not form a fused 6-membered ring containing two nitrogen atoms;
5) when G⁵ is OMe, G⁴ is OMe, X¹ is O, G¹ is SMe, both R^{x} is H, both R^{x} is H, and R^{z} is H, then G³ and G⁶ do not form a 5-membered ring containing the group —O—CH═N—; or
6) when G⁶ is H, G³ is OMe, X¹ is O, G¹ is OH or OMe, both R^{x} is H, both R^{y} is H, and R^{z} is H, then G⁴ and G⁵ do not form a 5-membered ring containing the group —OCH₂O—.

or a pharmaceutically acceptable salt, solvate, hydrate, crystalline form, non-crystalline form, or stereoisomer thereof.

The stereocenter of structure (I) indicated by “*” can be racemic, a mixture of R and S enriched in either the R or S isomer, in the R configuration, or in the S configuration. In one embodiment, the stereocenter of structure (I) indicated by “*” is in the S configuration.

In one embodiment, the (thio)colchicinoid compound comprises a compound according to structure (I) wherein G¹ is SR¹, SeR¹, TeR¹, PoR¹, P(R²)₂, N(R³)₂, N(R⁵)₂, or Si(R⁴)₃; specifically SR¹, SeR¹, TeR¹, P(R²)₂, N(R³)₂, or N(R⁵)₂; more specifically SR¹, SeR¹, or TeR¹; and still yet more specifically SR¹. Within this embodiment, R¹ is C₁-C₁₀ alkyl or C₁-C₁₀ haloalkyl, specifically R¹ is C₁-C₅ alkyl or C₁-C₅ haloalkyl, and more specifically R¹ is C₁-C₂ alkyl or C₁-C₂ haloalkyl. Also within this embodiment, each occurrence of R² is independently chosen from hydrogen, C₁-C₁₀ alkyl, C₃-C₇ cycloalkyl, or C₁-C₁₀ haloalkyl; specifically hydrogen, C₁-C₅ alkyl, or C₁-C₅ haloalkyl; and yet more specifically hydrogen, C₁-C₂ alkyl, or C₁-C₂ haloalkyl. Still within this embodiment, each occurrence of R³ independently is hydrogen, C₁-C₁₀ alkyl or C₃-C₇ cycloalkyl; specifically C₁-C₁₀ alkyl; more specifically C₁-C₅ alkyl; and yet more specifically C₁-C₂ alkyl.

In one embodiment, X¹ is O. In another embodiment, X¹ is S.

In one embodiment, R^{z} is hydrogen, halogen, C₁-C₃alkyl, or C₁-C₃haloalkyl; specifically hydrogen, fluoro, C₁-C₂alkyl, or C₁-C₂haloalkyl; more specifically hydrogen, C₁alkyl, or C₁haloalkyl; and still yet more specifically hydrogen.

In one embodiment, each occurrence of R^{x} is hydrogen, halogen, C₁-C₃alkyl, or C₁-C₃haloalkyl; specifically hydrogen, fluoro, C₁-C₂alkyl, or C₁-C₂haloalkyl; more specifically hydrogen, C₁alkyl, or C₁haloalkyl; and still yet more specifically hydrogen.

In one embodiment, each occurrence of R^{y} is hydrogen, halogen, C₁-C₃alkyl, or C₁-C₃haloalkyl; specifically hydrogen, fluoro, C₁-C₂alkyl, or C₁-C₂haloalkyl; more specifically hydrogen, C₁alkyl, or C₁haloalkyl; and still yet more specifically hydrogen.

In another embodiment, G² is —N(R^{b})₂ wherein a first R^{b} is hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, or C₂-C₆ alkanoyl; and the second R^{b} is hydrogen, C₁-C₅ alkyl, C₃-C₇ cycloalkyl, or C₁-C₅ haloalkyl; specifically where the second R^{b} is hydrogen, C₁-C₃ alkyl, or C₁-C₃ haloalkyl; and more specifically where the second R^{b} is hydrogen, C₁-C₂ alkyl, or C₁-C₂ haloalkyl. Within this embodiment, the first R^{b} is hydrogen, C₁-C₅ alkyl, C₃-C₇ cycloalkyl, C₁-C₅ haloalkyl, or C₂-C₆ alkanoyl; specifically hydrogen, C₁-C₃ alkyl, C₁-C₃ haloalkyl, or C₂-C₃ alkanoyl; and more specifically hydrogen, C₁-C₂ alkyl, C₁-C₂ haloalkyl, or C₂ alkanoyl.

In another embodiment, G² is —N(R^{b})₂ wherein the two R^{b} form a 5 or 6-membered cyclic group wherein the N is a member of the ring structure and further wherein the ring has 0, 1, or 2 additional heteroatoms chosen from N, O, or S. Exemplary G² groups according to this embodiment include morpholinyl, piperazinyl, piperidinyl, and pyrrolidinyl groups.

In one embodiment, G³, G⁴, or G⁵ is a carbohydrate group linked via an O-, N- or S-glycosidic bond; specifically G³ or G⁴; and more specifically G³. Exemplary carbohydrate or glycone groups include glucose, fructose, glucuronic acid, and the like having an α or β linkage, specifically a β linkage.

In another embodiment, G⁴ and G⁵ are each —OMe.

In yet another embodiment, G⁶ is H or —OMe.

In one embodiment, the compound of structure (I) is O, S, or N PEGylated at positions G′, G², G³, G⁴, or G⁵, specifically at G³ or G⁴, and more specifically at G³. Examples of a PEGylated derivative may involve the reaction of a PEGylating reagent with an appropriate reactive group on the (thio)colchicinoid core structure. Exemplary PEGylating reagents include those that pegylate hydroxyl, amine, or thiol groups such as mPEG-epoxide

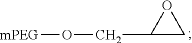

mPEG-NPC

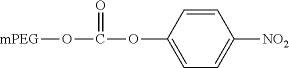

(mPEG-nitrophenyl carbonate); mPEG-isocyanate mPEG-O—CH₂CH₂—N═C═O, wherein the PEG weight average molecular weights (M_{w}) can be 400, 600, 800, 1000, 2000, 3400, or greater; mPEG-succinyl-NHS (“NHS”═N-succinimidyl group, MW 2000, 5000), mPEG-glutaryl-NHS (MW 5000), mPEG-carboxymethyl-NHS (MW 2000, 5000), and mPEG-carboxypentyl-NHS (MW 5000). Such reagents are commercially available from SunBio, Anyang City South Korea; or NOF Corporation, Tokyo Japan. The term “mPEG” means methoxy polyethylene glycol —O—CH₂CH₂—(OCH₂CH₂)ₓ—OCH₃.

In one embodiment, a compound according to structure (I) wherein G¹ is —SMe; X¹ is O; R^{x}, R^{y}, and R^{z} are all hydrogen; G² is —N(H)Ac; G⁴ is —OMe; G⁵ is —OMe and G³ is —OR^{a} wherein R^{a} is -L-PEG can be prepared by reacting 3-demethylthiocolchicine (CAS No. 87424-25-7) with a suitable PEGylation reagent such as mPEG-epoxide, mPEG-NPC, or mPEG-isocyanate using procedures well known in the art.

In one embodiment, G³ and G⁴ groups or G⁴ and G⁵ groups form a cyclic 5- or 6-membered ring having two O; specifically an acetonide prepared from 2,2-dimethoxypropane, para-toluene sulfonic acid and 2,3-didemethylthiocolchicine or 2,3-didemethylcolchicine in a suitable solvent. 2,3-Didemethylthiocolchicine and 2,3-didemethylcolchicine can be obtained using procedures described in U.S. Pat. No. 4,692,463 to Brossi.

In one embodiment, a compound according to structure (I) comprises a compound wherein G³ is —OH; G⁴ is —OMe; G⁵ is —OMe; G⁶ is H; R^{x} is hydrogen; R^{y} is hydrogen; R^{z} is hydrogen; G² is —N(H)Ac; X¹ is O; and G¹ is SeR¹, TeR¹, PoR¹, P(R²)₂, N(R³)₂, or Si(R⁴)₃; R¹ is hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl, wherein when R¹ is not hydrogen it is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; each occurrence of R² is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl; or the two R² form a 5- or 6-membered cyclic group having 0, 1, or 2 additional heteroatoms chosen from N, O, or S; wherein each occurrence of R² that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; each occurrence of R³ is independently chosen from C₈-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R³ form a 5- or 6-membered cyclic group having 0, 1, or 2 additional heteroatoms chosen from N, O, or S; wherein each occurrence of R³ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; wherein each occurrence of R⁴ independently is hydrogen, C₁-C₁₀ alkyl, C₂-C₁₀ alkenyl, or C₃-C₇ cycloalkyl; wherein each occurrence of R⁴ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy.

In another embodiment, a compound according to structure (I) comprises a compound wherein G³ is -L-PEG; G⁴ is —OMe; G⁵ is —OMe; G⁶ is H; R^{x} is hydrogen; R^{y} is hydrogen; R^{z} is hydrogen; G² is —N(H)Ac; X¹ is O; and G¹ is —SMe.

In yet another embodiment, a compound according to structure (I) comprises a compound wherein G³ is —OH; G⁴ is —OMe; G⁵ is —OMe; G⁶ is H; R^{x} is hydrogen; R^{y} is hydrogen; R^{z} is Me; G² is —N(H)Ac; X¹ is O; and G¹ is —SMe.

In another embodiment, the (thio)colchicinoid compound comprises a compound according to structure (II)

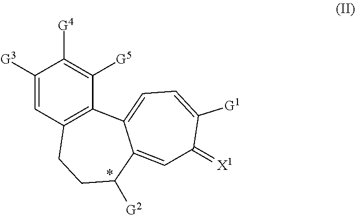

wherein G′, G², G³, G⁴, G⁵, and X¹ are as previously defined.

In yet another embodiment, the (thio)colchicinoid compound comprises a compound according to structure (III)

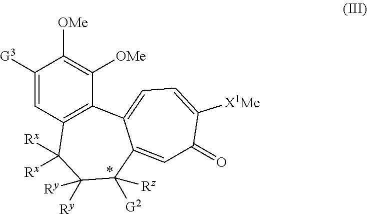

wherein G², G³, R^{x}, R^{y}, R^{z}, and X¹, are as previously defined.

In yet another embodiment, the (thio)colchicinoid compound comprises a compound according to structure (IV)

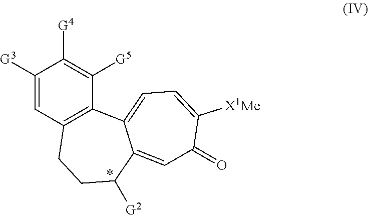

wherein G², G³, G⁴, G⁵, and X¹ are as previously defined.

In still yet another embodiment, the (thio)colchicinoid compound comprises a compound according to structure (V)

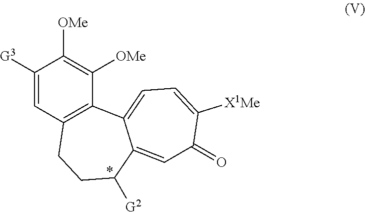

wherein G², G³, and X¹ are as previously defined.

In one embodiment, the (thio)colchicinoid compound comprises a compound according to structure (VI)

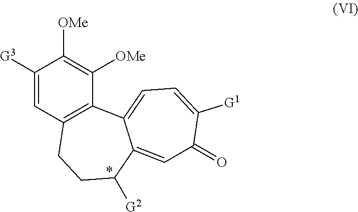

wherein G′, G², and G³ are as previously defined.

In another embodiment, the (thio)colchicinoid compound comprises a compound according to structure (VII)

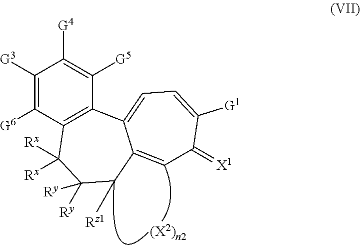

wherein G′, G³, G⁴, G⁵, G⁶, R^{x}, R^{y}, and X¹ are as previously defined and each occurrence of X² is independently O, N, NR^{b}, CR^{b}, or CR^{b}₂, to form a cyclic group (e.g. ═N—O— when n² is 2), where R^{b} is as previously defined, n² is 2 or 3, and R^{z1} is hydrogen or a bond forming the ring structure containing X²;

with the proviso that

1) when G⁶ is H, G³ is OMe, G⁴ is OMe, G⁵ is OMe, X¹ is O, G¹ is SMe, both R^{x} are H, and both R^{y} are H, then n² is not 2, R^{z1} is not a bond, and X² is not part of a 5-membered ring containing the group ═N—O— (where the oxygen is attached to the tropone ring),

2) when G⁶ is H, G³ is OMe, G⁴ is OMe, G⁵ is OMe, X¹ is O, G¹ is OMe, both R^{x} is H, and both R^{y} is H, then n² is not 2, R^{z1} is not H, and X² is not part of a 5-membered ring containing the group —N(Ac)—C(OH)(Me)— (where the carbon is attached to the tropone ring).

In another embodiment, the (thio)colchicinoid compound comprises a compound according to structure (VIII)

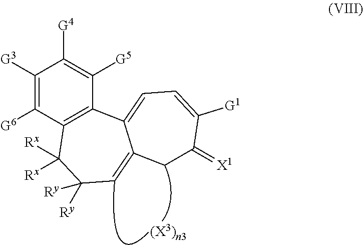

wherein G′, G³, G⁴, G⁵, G⁶, R^{x}, R^{y}, and X¹ are as previously defined and each occurrence of X³ is O, N, NR^{b}, CR^{b}, or CR^{b}₂ to form a cyclic group (e.g. —NH—CH═ or —NH—C(Me)═ when n³ is 2 (where the doublebond is bonded to the tropone ring)) where R^{b} is as previously defined, and n³ is 2 or 3;

with the proviso that when G⁶ is H, G³ is OMe, G⁴ is OMe, G⁵ is OMe, X¹ is O, G¹ is OMe, both R^{x} is H, and both R^{y} is H, then n³ is not 2 and X³ is not part of a 5-membered ring containing the group —NH—C(CHO)═ or —NH—C(Me)═ (where the doublebond is bonded to the tropone ring)).

In another embodiment, the (thio)colchicinoid compound comprises a compound according to structure (IX)

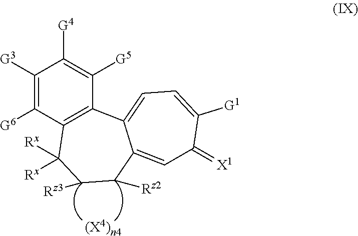

wherein G′, G³, G⁴, G⁵, G⁶, R^{x}, and X¹ are as previously defined and each instance of X⁴ is O, N, —NR^{b}C, CR^{b}, or CR^{b}₂, to form a saturated or unsaturated cyclic group optionally fused to a 5- or 6-membered cyclic or aryl group

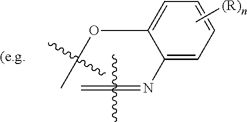

n is 0, 1, 2 or 3 and R is hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy) where R^{b} is as previously defined, n⁴ is 3 or 4, R^{z2} is hydrogen or a bond forming the ring structure containing X⁴, and R^{z3} is hydrogen, hydroxyl, or a bond forming the ring structure containing X⁴;

with the proviso that

1) when G⁶ is H, G³ is OMe, G⁴ is OMe, G⁵ is OMe, X¹ is O, G¹ is OMe or SMe, and both R^{x} is H, then n⁴ is not 4 and X⁴ is not part of a fused 6-membered ring containing the group

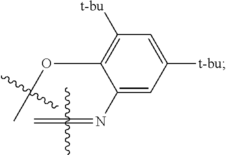

or

2) when G⁶ is H, G³ is OMe, G⁴ is OMe, G⁵ is OMe, X¹ is O, G¹ is OMe, and both R^{x} is H, then n⁴ is not 3 and X⁴ is not part of a 5-membered ring containing the group ═N—N(Ac)—CH═.

As used herein, “alkyl” includes straight chain and branched saturated aliphatic hydrocarbon groups, having the specified number of carbon atoms. Examples of alkyl include, but are not limited to, methyl, ethyl, n-propyl, isopropyl, n-butyl, 3-methylbutyl, t-butyl, n-pentyl, sec-pentyl, and n-hexyl. Specific alkyl groups include lower alkyl groups, those alkyl groups having 1, 2, 3, 4, 5, or 6 carbon atoms.

As used herein “cycloalkyl” are cyclic saturated aliphatic hydrocarbon groups, having the specified number of carbon atoms. Examples of cycloalkyl include, but are not limited to, cyclopentyl, cyclohexyl, and the like.

As used herein “haloalkyl” indicates straight chain and branched alkyl groups having the specified number of carbon atoms, substituted with 1 or more halogen atoms, generally up to the maximum allowable number of halogen atoms (“perhalogenated”, e.g. perfluorinated). Examples of haloalkyl include, but are not limited to, trifluoromethyl, difluoromethyl, 2-fluoroethyl, and penta-fluoroethyl.

As used herein, “alkoxy” includes an alkyl group as defined above with the indicated number of carbon atoms attached through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy.

“Haloalkoxy” indicates a haloalkyl group as defined above attached through an oxygen bridge.

As used herein, the term “aryl” indicates aromatic groups containing only carbon in the aromatic ring or rings. Such aromatic groups may be further substituted with carbon or non-carbon atoms or groups. Typical aryl groups contain 1 or 2 separate, fused, or pendant rings and from 6 to about 12 ring atoms, without heteroatoms as ring members. Where indicated aryl groups may be substituted. Such substitution may include fusion to a 5 to 7-membered saturated cyclic group that optionally contains 1 or 2 heteroatoms independently chosen from N, O, and S, to form, for example, a 3,4-methylenedioxy-phenyl group. Aryl groups include, for example, phenyl, naphthyl, including 1-naphthyl and 2-naphthyl, anthracene, pentacene, fluorene, and bi-phenyl.

“Halo” or “halogen” as used herein refers to fluoro, chloro, bromo, or iodo.

“Heteroaryl” indicates a stable 5- to 7-membered monocyclic or 7- to 10-membered bicyclic heterocyclic ring which contains at least 1 aromatic ring that contains from 1 to 4, or specifically from 1 to 3, heteroatoms chosen from N, O, and S, with remaining ring atoms being carbon. When the total number of S and O atoms in the heteroaryl group exceeds 1, these heteroatoms are not adjacent to one another. In one embodiment, the total number of S and O atoms in the heteroaryl group is not more than 2. Examples of heteroaryl groups include pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, and 5,6,7,8-tetrahydroisoquinoline.

“Heterocycloalkyl” is used to indicate saturated cyclic groups containing from 1 to about 3 heteroatoms chosen from N, O, and S, with remaining ring atoms being carbon. Heterocycloalkyl groups have from 3 to about 8 ring atoms, and more typically have from 5 to 7 ring atoms. A C₂-C₇heterocycloalkyl group contains from 2 to about 7 carbon ring atoms and at least one ring atom chosen from N, O, and S. Examples of heterocycloalkyl groups include morpholinyl, piperazinyl, piperidinyl, and pyrrolidinyl groups.

“Alkoxycarbonyl” indicates an alkoxy group, as defined above, having the indicated number of carbon atoms, attached through a keto linkage. The carbon of the carbonyl carbon is not included in the numbering, thus a C₂alkoxycarbonyl has the formula CH₃CH₂O(C═O)—.

“Alkanoyl” indicates an alkyl group attached through a keto (—(C═O)—) bridge. Alkanoyl groups have the indicated number of carbon atoms, with the carbon of the keto group being included in the numbered carbon atoms. For example a C₂alkanoyl group is an acetyl group having the formula CH₃(C═O)—.

“Alkylester” indicates an alkyl group attached through an ester linkage, i.e., attached through the oxygen —O(C═O)alkyl. Alkylester groups have the indicated number of carbon atoms with respect to the alkyl portion, with the carbon of the carbonyl group not being included in the numbering.

“Mono- or di-alkylamino” indicates secondary or tertiary alkyl amino groups, wherein the alkyl groups are as defined above and have the indicated number of carbon atoms. The point of attachment of the alkylamino group is on the nitrogen. Examples of mono- and di-alkylamino groups include ethylamino, dimethylamino, and methyl-propyl-amino.

“Alkenyl” as used herein indicates hydrocarbon chains of either a straight or branched configuration comprising one or more unsaturated carbon-carbon bonds, which may occur in any stable point along the chain, such as ethenyl and propenyl.

“Substituted” as used herein means that any one or more hydrogens on the designated atom or group is replaced with a selection from the indicated group, provided that the designated atom's normal valence is not exceeded.

A dash (“—”) that is not between two letters or symbols is used to indicate a point of attachment for a substituent. For example, —CHO is attached through carbon of the carbonyl group.

Compounds are described using standard nomenclature. For example, any position not substituted by any indicated group is understood to have its valency filled by a bond as indicated, or a hydrogen atom.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms (amorphous), and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Pharmaceutically acceptable salts” includes derivatives of an active agent, wherein the active agent is modified by making acid or base addition salts thereof, and further refers to pharmaceutically acceptable solvates, including hydrates, crystalline forms, non-crystalline forms, polymorphs, and stereoisomers of such salts. Examples of pharmaceutically acceptable salts include, but are not limited to, mineral or organic acid addition salts of basic residues such as amines; alkali or organic addition salts of acidic residues; and the like, or a combination comprising one or more of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, or a combination comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)_{z}—COOH where z is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; or a combination comprising one or more of the foregoing salts.

The (thio)colchicinoid compounds of structure (I) can be formulated for oral, buccal, sublingual, mucosal, transdermal, rectal, vaginal, subcutaneous, intramuscular, and intravenous delivery.

By “oral dosage form” is meant to include a unit dosage form for oral administration that may be solid, semisolid, or liquid. An oral dosage form may optionally comprise a plurality of subunits such as, for example, microcapsules or microtablets. Multiple subunits may be packaged for administration in a single dose. Other exemplary dosage forms for oral administration include, for example, suspension, an emulsion, an orally disintegrating tablet including an effervescent tablet, a sublingual tablet, an orally dissolving strip, a gastro-resistant tablet, a soft capsule, a hard capsule, a gastro-resistant capsule, a tablet, a coated granule, a gastro-resistant granule, and the like.

By “subunit” is meant to include a composition, mixture, particle, pellet, etc., that can provide an oral dosage form alone or when combined with other subunits.

Solid dosage forms for oral administration include, but are not limited to, capsules, tablets, powders, and granules. In such solid dosage forms, the active agent may be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (1 solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, or a combination comprising one or more of the foregoing additives. For capsules and tablets, the dosage forms may also comprise buffering agents.

Another suitable oral dosage form is a non-chewable, orally disintegrating tablet. These dosage forms can be made by methods known to those of ordinary skill in the art of pharmaceutical formulations. For example, Cima Labs has produced oral dosage forms including microparticles and effervescents, which rapidly disintegrate in the mouth and provide adequate taste-masking. Cima Labs has also produced a rapidly dissolving dosage form containing the active agent and a matrix that includes a nondirect compression filler and a lubricant. U.S. Pat. No. 5,178,878 and U.S. Pat. No. 6,221,392 provide teachings regarding orally disintegrating tablets.

An exemplary orally disintegrating tablet includes a mixture incorporating a water or saliva activated effervescent disintegration agent and subunits such as coated particles, specifically of a size such that chewing does not damage the structure of the subunit. The mixture including the subunits and effervescent disintegration agent may be formulated as a tablet of a size and shape adapted for direct oral administration to a patient. The tablet is substantially completely disintegrable upon exposure to water or saliva. The effervescent disintegration agent is present in an amount effective to aid in disintegration of the tablet, and to provide a distinct sensation of effervescence when the tablet is placed in the mouth of a patient.

The effervescent sensation is not only pleasant to the patient but also tends to stimulate saliva production, thereby providing additional water to aid in further effervescent action. Thus, once the tablet is placed in the patient's mouth, it will disintegrate rapidly and substantially completely without any voluntary action by the patient. Even if the patient does not chew the tablet, disintegration will proceed rapidly. Upon disintegration of the tablet, the subunits are released and can be swallowed as a slurry or suspension. The subunits thus may be transferred to the patient's stomach for dissolution in the digestive tract and systemic distribution of the active agent.

The term effervescent disintegration agent includes compounds which evolve gas. The preferred effervescent disintegration agents evolve gas by means of chemical reactions which take place upon exposure of the effervescent disintegration agent to water or to saliva in the mouth. The bubble or gas generating reaction is most often the result of the reaction of a soluble acid source and an alkali metal carbonate or carbonate source. The reaction of these two general classes of compounds produces carbon dioxide gas upon contact with water included in saliva.

Such water activated materials may be kept in a generally anhydrous state with little or no absorbed moisture or in a stable hydrated form since exposure to water will prematurely disintegrate the tablet. The acid sources or acid may be those which are safe for human consumption and may generally include food acids, acid anhydrides and acid salts. Food acids include citric acid, tartaric acid, malic acid, fumaric acid, adipic acid, and succinic acids etc. Because these acids are directly ingested, their overall solubility in water is less important than it would be if the effervescent tablet formulations were intended to be dissolved in a glass of water. Acid anhydrides and acid of the above described acids may also be used. Acid salts may include sodium, dihydrogen phosphate, disodium dihydrogen pyrophosphate, acid citrate salts and sodium acid sulfite.

Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, or a combination comprising at least one of the foregoing carbonates.

The effervescent disintegration agent is not always based upon a reaction which forms carbon dioxide. Reactants which evolve oxygen or other gasses which are safe are also considered within the scope. Where the effervescent agent includes two mutually reactive components, such as an acid source and a carbonate source, it is preferred that both components react substantially completely. Therefore, an equivalent ratio of components which provides for equal equivalents is preferred. For example, if the acid used is diprotic, then either twice the amount of a mono-reactive carbonate base, or an equal amount of a di-reactive base should be used for complete neutralization to be realized. However, the amount of either acid or carbonate source may exceed the amount of the other component. This may be useful to enhance taste or performance of a tablet containing an overage of either component. In this case, it is acceptable that the additional amount of either component may remain unreacted.

In general, the amount of effervescent disintegration agent useful for the formation of tablets is about 5 wt % to about 50 wt % based on the total weight of the final dosage form, specifically about 15 wt % and about 30 wt %, and more specifically about 20 wt % to about 25 wt %.

Other types of orally disintegrating tablets can be prepared without an effervescent agent by using a spray dried carbohydrate or sugar alcohol excipients (e.g. sorbitol, mannitol, xylitol, or a combination comprising at least one of the foregoing, and the like), optionally combined with a disintegrant (e.g. the disintegrant is selected from crospovidone, croscarmellose, sodium starch glycolate, pregelatinized starch, partially pregelatinized starch, or a combination comprising at least one of the foregoing, and the like), or a glidant (e.g. colloidal silica, silica gel, precipitated silica, or a combination comprising at least one of the foregoing, and the like). Suitable orally disintegrating tablets can be found in U.S. Patent Application Publication US20030118642 A1 to Norman et al. incorporated herein in its entirety.

Orally disintegrating tablets can be manufactured by well-known tableting procedures. In common tableting processes, the material which is to be tableted is deposited into a cavity, and one or more punch members are then advanced into the cavity and brought into intimate contact with the material to be pressed, whereupon compressive force is applied. The material is thus forced into conformity with the shape of the punches and the cavity.

The orally disintegrating tablets typically rapidly disintegrate when orally administered. By “rapid”, it is understood that the tablets disintegrate in the mouth of a patient in less than about 7 minutes, and specifically between about 30 seconds and about 5 minutes, specifically the tablet should dissolve in the mouth between about 45 seconds and about 2 minutes. Disintegration time in the mouth can be measured by observing the disintegration time of the tablet in water at about 37° C. The tablet is immersed in the water without forcible agitation. The disintegration time is the time from immersion for substantially complete dispersion of the tablet as determined by visual observation. As used herein, the term “complete disintegration” of the tablet does not require dissolution or disintegration of the subunits or other discrete inclusions. In one embodiment, disintegration can be determined by USP 32 (Test <701>).

In another embodiment, the orally disintegrating tablets include those having a dissolution rate of more than 65% release of active agent within 15 minutes. A dissolution profile is a plot of the cumulative amount of active agent released as a function of time. A dissolution profile can be measured, for example, utilizing the standard test for dissolution according to USP 32 (Test <711>) or Drug Release Test <724>. A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

In another embodiment, the (thio)colchicinoid compounds are formulated into an orally dissolving strip, which rapidly dissolves in the mouth to release the active agent within the strip. The orally dissolving strips generally comprise a water soluble polymer and a (thio)colchicinoid compound. Exemplary classes of water soluble polymers include water soluble cellulosic polymers, water soluble synthetic polymers, water soluble natural gums and polymers or derivatives thereof, or a combination comprising at least one of the foregoing. Exemplary water soluble cellulosic polymers include hydroxypropyl cellulose, hydroxypropylmethyl cellulose, hydroxyethyl cellulose, carboxymethyl cellulose, or a combination comprising at least one of the foregoing. Exemplary water soluble natural gums and polymers include amylose, dextran, casein, pullulan, gelatin, pectin, agar, carrageenan, xanthan gum, tragacanth, guar gum, acacia gum, arabic gum, sodium alginate, zein, or a combination comprising at least one of the foregoing. Exemplary water soluble synthetic polymers include polyethylene glycol, polyethylene oxide, polyvinyl pyrrolidone, polyvinyl alcohol, carboxyvinyl polymers, water soluble polyacrylic acid/acrylate, or a combination comprising at least one of the foregoing.

The water soluble polymer may be present in amounts of about 20 to about 95, specifically about 30 to about 85, and more specifically about 40 to about 75 wt % based on the total weight of the orally dissolving strip.

The orally dissolving strip can further optionally comprise a plasticizer in addition to the water soluble polymer and active agent. Exemplary plasticizers include propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol, or a combination comprising at least one of the foregoing. The plasticizer may be present in amounts of about 0 to about 20, specifically about 1 to about 15, and more specifically about 5 to about 10 wt % based on the total weight of the orally dissolving strip.

The orally dissolving strip can further optionally comprise an emulsifying agent in addition to the water soluble polymer and active agent. Exemplary emulsifying agents include polyvinyl alcohol, a sorbitan esters, a cyclodextrin, benzyl benzoate, glyceryl monostearate, a polyoxyethylene alkyl ether, a polyoxyethylene stearate, poloxamer, a polyoxyethylene castor oil derivative, a hydrogenated vegetable oil, a polysorbate, or a combination comprising at least one of the foregoing

The emulsifying agent may be present in amounts of about 0 to about 20, specifically about 1 to about 15, and more specifically about 5 to about 10 wt % based on the total weight of the orally dissolving strip.

The orally dissolving strip can further optionally comprise a flavor or sweetener in addition to the water soluble polymer and active agent. Exemplary sweeteners include sugar, a monosaccharide, an oligosaccharide, aldose, ketose, dextrose, maltose, lactose, glucose, fructose, sucrose, a sugar polyol (e.g., mannitol, xylitol, sorbitol, erythritol, and the like), artificial sweeteners (e.g., acesulfame potassium, sucralose, aspartame, saccharin, sodium saccharin, and the like) or a combination comprising at least one of the foregoing. The sweetener may be present in amounts of about 0 to about 20, specifically about 1 to about 15, and more specifically about 5 to about 10 wt % based on the total weight of the orally dissolving strip.

In some embodiments, the orally dissolving formulations of the present invention may comprise an excipient. Suitable excipients include, but are not limited to, microcrystalline cellulose, colloidal silicon dioxide, talc, starch, or a combination comprising at least one of the foregoing. In some embodiments, the excipient may include talc as anti-adhering agent.

Other optional components that can be used to prepare the orally dissolving strip include a filler/diluent, a surfactant, a disintegrating agent, an antifoaming agent, an antioxidant, a buffering agent, a color, or a combination comprising at least one of the foregoing.

In one embodiment, particles of the (thio)colchicinoid compound are coated with a taste-masking polymer for greater patient acceptability. Exemplary taste-making polymers include meth/acrylic and meth/acrylate polymers and copolymers such as Eudragit® polymers from Evonik Industries (amino methacrylate copolymer, Eudragit® E PO, E 100, and E 12,5; and methacrylic acid copolymer Type A, B, and C, Eudragit® L 100, S 100, and L 100-55). Other taste-masking polymers include cellulose acetate phthalate, ethyl vinyl phthalate, polyvinyl acetate phthalate, a hydroxy alkyl cellulose phthalate, or a combination comprising at least one of the foregoing.

The taste-masking polymer can be used in an amount of about 1 to about 35 wt % based on the total weight of active agent and taste-masking polymer, specifically about 3 to about 20 wt %, and more specifically about 5 to about 10 wt %.

In one embodiment, the orally dissolving strip exhibits a drug loading of not more than 50% w/w of the film. Exemplary orally dissolving strips will comprise about 0.01 to about 50 mg of active agent per strip. In another embodiment, the orally dissolving strip has a thickness of about 0.1 to about 5.0 millimeters, specifically about 0.3 to about 4.0 and yet more specifically about 0.5 to about 2.5. In another embodiment the orally dissolving strip has a surface area of about 1.0 to about 6.0, specifically about 1.2 to about 4.0 and yet more specifically about 1.5 to about 2.0 square centimeters.

The orally dissolving strip once placed in the oral cavity may dissolve after less than about 60 seconds, specifically less than 30 seconds, and yet more specifically less than about 20 seconds.

A solvent can be used in the process to prepare the orally dissolving strip, including water, ethanol, 1-butanol, 2-butanol, 2-ethoxyethanol, ethyl acetate, methyl acetate, 3-methyl-1-butanol, methylethyl ketone, 2-methyl-1-propanol, isobutyl acetate, isopropyl acetate ethyl ether, tert-butylmethyl ether acetone, or a combination comprising at least one of the foregoing. The solvent is used for processing and then removed to result in the final product.

Methods of preparing orally dissolving strips involve solvent casting and film coating. The active agent is mixed with film-forming excipients and solvents such as water, ethanol, and the like. A thin coating of the mixture is cast on a moving, inert substrate and the coated substrate is moved through a drying oven to evaporate the solvent before die-cutting the dried film into strips. Another method involves hot-melt extrusion, by melting an active agent and excipient polymer blend which is then extruded through a die under molten conditions. The thin film is then cooled to room temperature and die-cut into strips.

The (thio)colchicinoid compounds disclosed herein are suitable for treating a patient in need thereof, specifically for use as a non-sedating muscle relaxant. The (thio)colchicinoid compounds can also be used as an anti-inflammatory agent, an anti-gout agent, and anti-cancer agent, or an anti-proliferative agent. The (thio)colchicinoid compound is administered in an amount sufficient to provide the desired therapeutic effect (e.g., muscle relaxant activity) to the patient. Amounts can be determined by the skilled artisan using techniques known in the art. Exemplary amounts of (thio)colchicinoid compounds can be about 0.01 to about 50 mg per day, specifically about 1 to about 40 mg per day, more specifically about 4 to about 30 mg per day, and yet more specifically about 8 to about 20 mg per day.

In another embodiment, the (thio)colchicinoid compounds can be used as reference compounds for use in various analytical methods, particularly the compounds where G¹ comprises a selenium, tellurium, or a polonium element.

Exemplary compounds according to structure (I) include those provided in Table A where R^{x}, R^{y}, and R^{z}=hydrogen, Ac=acetyl.

(I)

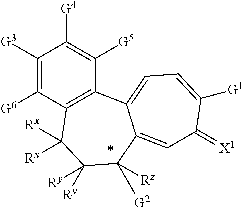

| No. | G¹ | X¹ | G² | G³ | G⁴ | G⁵ | G⁶ |
|---|---|---|---|---|---|---|---|
| 1 | —SeMe | O | —N(H)Ac | —OMe | —OMe | —OMe | H |
| 2 | —TeMe | O | —N(H)Ac | —OMe | —OMe | —OMe | H |
| 3 | —PoMe | O | —N(H)Ac | —OMe | —OMe | —OMe | H |
| 4 | —N(H)(CH₂)₈CH₃ | O | —N(H)Ac | —OMe | —OMe | —OMe | H |
| 5 | —P(Me)₂ | O | —N(H)Ac | —OMe | —OMe | —OMe | H |
| 6 | —Si(Me)₃ | O | —N(H)Ac | —OMe | —OMe | —OMe | H |
| 7 | —SeMe | S | —N(H)Ac | —OMe | —OMe | —OMe | H |
| 8 | —TeMe | S | —N(H)Ac | —OMe | —OMe | —OMe | H |
| 9 | —PoMe | S | —N(H)Ac | —OMe | —OMe | —OMe | H |
| 10 | —N(H)(CH₂)₈CH₃ | S | —N(H)Ac | —OMe | —OMe | —OMe | H |
| 11 | —P(Me)₂ | S | —N(H)Ac | —OMe | —OMe | —OMe | H |
| 12 | —Si(Me)₃ | S | —N(H)Ac | —OMe | —OMe | —OMe | H |
| 13 | —SeMe | O | —N(H)Ac | —OH | —OMe | —OMe | H |
| 14 | —TeMe | O | —N(H)Ac | —OH | —OMe | —OMe | H |
| 15 | —PoMe | O | —N(H)Ac | —OH | —OMe | —OMe | H |
| 16 | —N(H)(CH₂)₈CH₃ | O | —N(H)Ac | —OH | —OMe | —OMe | H |
| 17 | —P(Me)₂ | O | —N(H)Ac | —OH | —OMe | —OMe | H |
| 18 | —Si(Me)₃ | O | —N(H)Ac | —OH | —OMe | —OMe | H |
| 19 | —SeMe | S | —N(H)Ac | —OH | —OMe | —OMe | H |
| 20 | —TeMe | S | —N(H)Ac | —OH | —OMe | —OMe | H |
| 21 | —PoMe | S | —N(H)Ac | —OH | —OMe | —OMe | H |
| 22 | —N(H)(CH₂)₈CH₃ | S | —N(H)Ac | —OH | —OMe | —OMe | H |
| 23 | —P(Me)₂ | S | —N(H)Ac | —OH | —OMe | —OMe | H |
| 24 | —Si(Me)₃ | S | —N(H)Ac | —OH | —OMe | —OMe | H |
| 25 | —SeMe | O | —N(H)Ac | β-D-glucopyranosyloxy | —OMe | —OMe | H |
| 26 | —TeMe | O | —N(H)Ac | β-D-glucopyranosyloxy | —OMe | —OMe | H |
| 27 | —PoMe | O | —N(H)Ac | β-D-glucopyranosyloxy | —OMe | —OMe | H |
| 28 | —N(H)(CH₂)₈CH₃ | O | —N(H)Ac | β-D-glucopyranosyloxy | —OMe | —OMe | H |
| 29 | —P(Me)₂ | O | —N(H)Ac | β-D-glucopyranosyloxy | —OMe | —OMe | H |
| 30 | —Si(Me)₃ | O | —N(H)Ac | β-D-glucopyranosyloxy | —OMe | —OMe | H |
| 31 | —SeMe | S | —N(H)Ac | β-D-glucopyranosyloxy | —OMe | —OMe | H |
| 32 | —TeMe | S | —N(H)Ac | β-D-glucopyranosyloxy | —OMe | —OMe | H |
| 33 | —PoMe | S | —N(H)Ac | β-D-glucopyranosyloxy | —OMe | —OMe | H |
| 34 | —N(H)(CH₂)₈CH₃ | S | —N(H)Ac | β-D-glucopyranosyloxy | —OMe | —OMe | H |
| 35 | —P(Me)₂ | S | —N(H)Ac | β-D-glucopyranosyloxy | —OMe | —OMe | H |
| 36 | —Si(Me)₃ | S | —N(H)Ac | β-D-glucopyranosyloxy | —OMe | —OMe | H |
| 37 | —SeMe | O | —N(H)Ac | β-D- | —OMe | —OMe | H |
| | | | | glucopyranosiduronic | | | |
| | | | | acid | | | |
| 38 | —TeMe | O | —N(H)Ac | β-D- | —OMe | —OMe | H |
| | | | | glucopyranosiduronic | | | |
| | | | | acid | | | |
| 39 | —PoMe | O | —N(H)Ac | β-D- | —OMe | —OMe | H |
| | | | | glucopyranosiduronic | | | |
| | | | | acid | | | |
| 40 | —N(H)(CH₂)₈CH₃ | O | —N(H)Ac | β-D | —OMe | —OMe | H |
| | | | | glucopyranosiduronic | | | |
| | | | | acid | | | |
| 41 | —P(Me)₂ | O | —N(H)Ac | β-D- | —OMe | —OMe | H |
| | | | | glucopyranosiduronic | | | |
| | | | | acid | | | |
| 42 | —Si(Me)₃ | O | —N(H)Ac | β-D- | —OMe | —OMe | H |
| | | | | glucopyranosiduronic | | | |
| | | | | acid | | | |
| 43 | —SeMe | S | —N(H)Ac | β-D- | —OMe | —OMe | H |
| | | | | glucopyranosiduronic | | | |
| | | | | acid | | | |
| 44 | —TeMe | S | —N(H)Ac | β-D- | —OMe | —OMe | H |
| | | | | glucopyranosiduronic | | | |
| | | | | acid | | | |
| 45 | —PoMe | S | —N(H)Ac | β-D- | —OMe | —OMe | H |
| | | | | glucopyranosiduronic | | | |
| | | | | acid | | | |
| 46 | —N(H)(CH₂)₈CH₃ | S | —N(H)Ac | β-D- | —OMe | —OMe | H |
| | | | | glucopyranosiduronic | | | |
| | | | | acid | | | |
| 47 | —P(Me)₂ | S | —N(H)Ac | β-D- | —OMe | —OMe | H |
| | | | | glucopyranosiduronic | | | |
| | | | | acid | | | |
| 48 | —Si(Me)3 | S | —N(H)Ac | β-D- | —OMe | —OMe | H |
| | | | | glucopyranosiduronic | | | |
| | | | | acid | | | |
| 49 | —SMe | O | —N(H)Ac | mPEG—O—CH₂—CH(OH)—CH₂—O-(Mw 400) | —OMe | —OMe | H |
| 50 | —SMe | O | —N(H)Ac | mPEG—O—CH₂—CH(OH)—CH₂—O-(Mw 600) | —OMe | —OMe | H |
| 51 | —SMe | O | —N(H)Ac | mPEG—O—CH₂—CH(OH)—CH₂—O-(Mw 800) | —OMe | —OMe | H |
| 52 | —SMe | O | —N(H)Ac | mPEG—O—CH₂—CH(OH)—CH₂—O-(Mw 1000) | —OMe | —OMe | H |
| 53 | —SMe | O | —N(H)Ac | mPEG—O—CH₂—CH(OH)—CH₂—O-(Mw 2000) | —OMe | —OMe | H |
| 54 | —SMe | O | —N(H)Ac | mPEG—O—C(═O)—O-(Mw 400) | —OMe | —OMe | H |
| 55 | —SMe | O | —N(H)Ac | mPEG—O—C(═O)—O-(Mw 600) | —OMe | —OMe | H |
| 56 | —SMe | O | —N(H)Ac | mPEG—O—C(═O)—O-(Mw 800) | —OMe | —OMe | H |
| 57 | —SMe | O | —N(H)Ac | mPEG—O—C(═O)—O-(Mw 1000) | —OMe | —OMe | H |
| 58 | —SMe | O | —N(H)Ac | mPEG—O—C(═O)—O-(Mw 2000) | —OMe | —OMe | H |
| 59 | —SMe | O | —N(H)Ac | mPEG—O—CH₂CH₂—NH—C(═O)—O-(Mw 400) | —OMe | —OMe | H |
| 60 | —SMe | O | —N(H)Ac | mPEG—O—CH₂CH₂—NH—C(═O)—O-(Mw 600) | —OMe | —OMe | H |
| 61 | —SMe | O | —N(H)Ac | mPEG—O—CH₂CH₂—NH—C(═O)—O-(Mw 800) | —OMe | —OMe | H |
| 62 | —SMe | O | —N(H)Ac | mPEG—O—CH₂CH₂—NH—C(═O)—O-(Mw 1000) | —OMe | —OMe | H |
| 63 | —SMe | O | —N(H)Ac | mPEG—O—CH₂CH₂—NH—C(═O)—O-(Mw 2000) | —OMe | —OMe | H |
| 64 | —SMe | O | —N(H)Ac | mPEG—O—C(═O)—NH-(Mw 400) | —OMe | —OMe | H |
| 65 | —SMe | O | —N(H)Ac | mPEG—O—C(═O)—NH-(Mw 2000) | —OMe | —OMe | H |
| 66 | —SMe | O | —N(H)Ac | mPEG—O—CH₂CH₂—NH—C(═O)—NH-(Mw 400) | —OMe | —OMe | H |
| 67 | —SMe | O | —N(H)Ac | mPEG—O—CH₂CH₂—NH—C(═O)—NH-(Mw 2000) | —OMe | —OMe | H |
| 68 | —SMe | O | —N(H)Ac | mPEG—O—C(═O)—CH₂CH₂—C(═O)NH- | —OMe | —OMe | H |
| | | | | (Mw 2000) | | | |
| 69 | —SMe | O | —N(H)Ac | mPEG—O—C(═O)—CH₂CH₂—C(═O)NH- | —OMe | —OMe | H |
| | | | | (Mw 5000) | | | |

| | | | | | | |
|---|---|---|---|---|---|---|
| 70 | —P(Me)₂ | O | —N(H)Ac | —O—C(Me)₂—O— | —OMe | H |
| 71 | —P(Me)₂ | O | —N(H)Ac | —O—CH₂—O— | —OMe | H |

| | | | | | | |
|---|---|---|---|---|---|---|
| 72 | —P(Me)2 | O | —N(H)Ac | —OH | —O—CH₂—O— | H |
| 73 | —P(Me)2 | O | —N(H)Ac | —OH | —O—C(Me)₂—O— | H |

The following examples further illustrate the invention but, of course, should not be construed as in any way limiting its scope.

### EXAMPLES

### Preparation of 3-demethylthiocolchicine (CAS No. 87424-25-7)

3-Demethylthiocolchicine can be prepared from 3-demethylcolchicine by the reaction with sodium or potassium methylthiolate in water at temperatures between 0 to 30° C., specifically room temperature. 3-Demethylcolchicine can be obtained from plant species Colchicum autumnale. Alternatively, 3-demethylthiocolchicine can be prepared from the hydrolysis of thiocolchicoside with 80% phosphoric acid. Sharma et al. Heterocycles, 1983, Vol. 20, p. 1587. 3-Demethylthiocolchicine can be prepared from the procedure of Example 1 of U.S. Pat. No. 5,175,342, the specific example incorporated by reference herein.

### Preparation of 2,3-didemethylthiocolchicine and 2,3-didemethylcolchicine

2,3-Didemethylthiocolchicine and 2,3-didemethylcolchicine can be obtained using procedures described in U.S. Pat. No. 4,692,463 to Brossi.

### Example 1

### 3-[mPEG]-3-demethylthiocolchicine carbonate

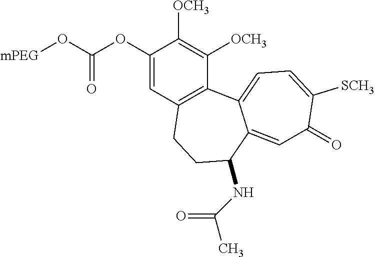

3-Demethylthiocolchicine (100 mg) is dissolved in toluene (5 ml) and mPEG-nitrophenyl carbonate (PEG Mw of about 400) (1 eq.) is added to the reaction flask. The reaction is allowed to stir, optionally with moderate heating, for twelve hours. The reaction mixture is then diluted with methylene chloride, washed with dilute HCl, and then brine. The organic layer is dried over Na₂SO₄ or MgSO₄, filtered to remove the drying agent, and stripped of solvent to yield 3-[mPEG]-3-demethylthiocolchicine carbonate.

### Example 2

### 3-[mPEG]-2,3-didemethylthiocolchicine carbonate

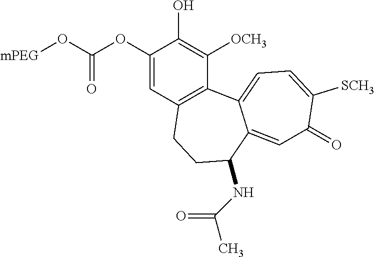

3-[mPEG]-2,3-didemethylthiocolchicine carbonate can be prepared similarly to Example 1 using 2,3-didemethylthiocolchicine carbonate.

### Example 3

### Determination of Muscle Relaxant Activity, Method A or B

### Method A

Muscle relaxant activity is evaluated by recording the polysynaptic reflex in normal rats. Male Sprague-Dawley rats (200-500 g) are treated with intraperitoneal doses of thiocolchicine derivative in distilled water with a drop of 0.05% Tween-80 at doses of 3, 10, and 30 mg/kg. The flexor reflex is elicited by electric shocks to the sural nerve innervation area in the right hindpaw of the rats via stainless steel needles inserted subcutaneously. The flexor reflex is recorded as electromyogram activity via needle electrodes inserted in the ipsilateral posterior biceps femoris/semitendinosus muscles. The stimulus parameters are 0.2-0.5 ms and 0.2 Hz and activates mainly Aδ fibers. The stimulus intensity is defined by the recording conditions within each experiment such that the stimulus is sufficient to produce a stable response (8-12 mA). The electromyogram is amplified, filtered (10-1000 Hz), rectified and integrated within a time-window from 8-10 until 35-40 ms after the stimulus. The magnitude of these reflexes after drug administration is expressed as a percentage of the mean value of the predrug control period. A two-way ANOVA and Dunnett's test are performed. Thiocolchicoside is used as the reference compound.

### Method B

Muscle relaxant activity is evaluated with the rota-rod test. Swiss male mice weighing 20-25 g are treated intraperitoneally with a (thio)colchicinoid compound at doses of 1, 3, and 10 mg/kg, thirty minutes before the test. Relaxant activity on striated muscles is evaluated by testing the resistance of the mice to the stimuli of a rotating plane revolving at increasing rate, from 2 to 50 r.p.m. Thiocolchicoside is used as the reference compound.

### Example 4

### Orally Disintegrating Tablet

A spray-dried mixture (550 grams) of mannitol and sorbitol in an 80:20 ratio (SPI Pharma Inc.), 61.00 grams of crospovidone and 1.5 grams of colloidal silicon dioxide are blended. The (thio)colchicinoid compound of Example 1 (400 grams) is then blended with the excipient mixture, optionally with flavor, color, high intensity sweetener, and food acid. Finally, magnesium stearate or sodium stearyl fumarate (16 grams) is added and blended. The final mixture is tableted using standard tableting procedures. The final mixture can be compressed into tablets using low force (4-20 kN) to form the orally disintegrating tablets. The orally disintegrating tablet dissolves in the oral cavity in less than seven minutes.

Orally disintegrating tablet containing the (thio)colchicinoid compounds of Examples 1 to 2 can be prepared in similar fashion.

### Example 5

### Orally Dissolving Strip

An orally dissolving strip is prepared by dissolving polyethylene oxide in water followed by the addition of polyethylene glycol (plasticizer), a sweetening agent (acesulfame potassium), sodium citrate, and polyoxyl castor oil. The (thio)colchicinoid compound of Example 1 is then added and mixed before casting a film on a Teflon surface using a BYK-Gardner film casting knife. The film is dried in an oven at a temperature of about 50-80° C. until dried. The dried film is then cut to size. The orally dissolving strip dissolves in the oral cavity in less than 60 seconds.

Orally dissolving strips containing the (thio)colchicinoid compounds of Examples 1 to 2 can be prepared in similar fashion.

The terms “comprising”, “having”, “including”, and “containing” are to be construed as open-ended terms (i.e., meaning “including, but not limited to”). The terms “a” and “an” do not denote a limitation of quantity, but rather denote the presence of at least one of the referenced item. The term “or” means “and/or”. The endpoints of all ranges directed to the same component or property are inclusive and independently combinable.

Unless defined otherwise, technical and scientific terms used herein have the same meaning as is commonly understood by one of skill in the art to which this invention belongs.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A compound according to structure (I) 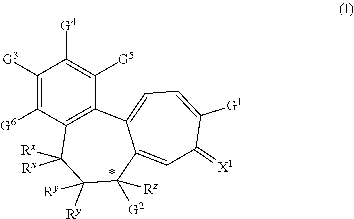
wherein
A) when G¹ is SeR¹, TeR¹, PoR¹, P(R²)₂, N(R³)₂, or Si(R⁴)₃;
R¹ is hydrogen, C₁-C₁₀alkyl, C₂-C₁₀ alkenyl, C₃-C₇ cycloalkyl, C₁-C₁₀haloalkyl, heterocycloalkyl, aryl, or heteroaryl, wherein when R¹ is not hydrogen it is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R² is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl; or the two R² form a 5- or 6-membered cyclic group having 0, 1, or 2 additional heteroatoms chosen from N, O, or S; wherein each occurrence of R² that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R³ is independently chosen from C₈-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R³ form a 5- or 6-membered cyclic group having 0, 1, or 2 additional heteroatoms chosen from N, O, or S; wherein each occurrence of R³ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
wherein each occurrence of R⁴ independently is hydrogen, C₁-C₁₀ alkyl, C₂-C₁₀ alkenyl, or C₃-C₇ cycloalkyl; wherein each occurrence of R⁴ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
X¹ is O or S;
G² is —OH, —OR^{a}, —SR^{a}, —N(R^{b})₂, heterocycloalkyl, aryl, heteroaryl, or a carbohydrate group linked via an O-, N- or S-glycosidic bond;
wherein R^{a} is C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, epoxide, carbonate, or N-succinimidyl group; wherein R^{a} is optionally substituted with 1, 2, or 3 substituents independently chosen from hydroxy, amino, cyano, halogen, C₁-C₆alkyl, C_{z}—C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
wherein each occurrence of R^{b} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C_{z}—C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{b} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{b} form a 5- or 6-membered cyclic group having 0, 1, or 2 additional heteroatoms chosen from N, O, or S;
each occurrence of R^{x}, R^{y}, and R^{z} is independently chosen from hydrogen, halogen, C₁-C₃alkyl, or C₁-C₃haloalkyl, or
where the two R^{x} groups, the two R^{y} groups, one R^{x} and one R^{y} group, or one R^{y} and R^{z} group form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
G³ is —OH, —OR^{a}, —N(R^{c})₂, heterocycloalkyl, aryl, heteroaryl, or a carbohydrate group linked via an O-, N- or S-glycosidic bond;
wherein each occurrence of R^{c} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{c} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{c} form a 5- or 6-membered cyclic group having 0, 1, or 2 additional heteroatoms chosen from N, O, or S;
G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁵ is —OH, —OR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁶ is H, —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined; or where the G³ and G⁴ groups or G⁴ and G⁵ groups or G³ and G⁶ groups form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
B) wherein when at least one of G², G³, G⁴, G⁵ or G⁶ is —OR^{a}, —SR^{a}, —N(R^{b})₂, or —N(R^{c})₂ wherein a R^{a}, a R^{b} or a R^{c} is -L-PEG, then the remaining G², G³, G⁴, G⁵ and G⁶ are as previously defined;
G¹ is OR¹, SR¹, SeR¹, TeR¹, PoR¹, P(R²)₂, N(R⁵)₂, or Si(R⁴)₃, where R¹, R² and R⁴ are as previously defined;
wherein each occurrence of R⁵ is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R⁵ form a 5- or 6-membered cyclic group having 0, 1, or 2 additional heteroatoms chosen from N, O, or S; wherein each occurrence of R⁵ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
X¹ is O or S; and
R^{x}, R^{y}, and R^{z} are as previously defined;
C) wherein when at least one of R^{x}, R^{y}, and R^{z} is not hydrogen, then the remaining R^{x}, R^{y}, and R^{z} are as previously defined;
G¹ is OR¹, SR¹, SeR¹, TeR¹, PoR¹, P(R²)₂, N(R⁵)₂, or Si(R⁴)₃, where R¹, R², R⁴ and R⁵ are as previously defined;
X¹ is O or S;
G² is —OH, —OR^{a}, —SR^{a}, —N(R^{b})₂ heterocycloalkyl, aryl, heteroaryl, or a carbohydrate group linked via an O-, N- or S-glycosidic bond, wherein R^{a} and R^{b} are as previously defined;
G³ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined; and
G⁵ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁶ is H, —OH, —OR¹, —SR^{a}, —N(R^{c})₂, heterocycloalkyl, aryl, heteroaryl, or a carbohydrate group linked via an O-, N- or S-glycosidic bond, and R^{a} and R^{c} are as previously defined; or
D) wherein
G¹ is OR¹ or SR¹;
R¹ is hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl, wherein when R¹ is not hydrogen it is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
X¹ is O or S;
G² is —OH, —OR^{a}, —SR^{a}, —N(R^{b})₂, heterocycloalkyl, aryl, heteroaryl, or a carbohydrate group linked via an O-, N- or S-glycosidic bond;
wherein R^{a} is C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, epoxide, carbonate, or N-succinimidyl group; wherein R^{a} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
wherein each occurrence of R^{b} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{b} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{b} form a 5- or 6-membered cyclic group having 0, 1, or 2 additional heteroatoms chosen from N, O, or S;
each occurrence of R^{x}, R^{y}, and R^{z} is independently chosen from hydrogen, halogen, C₁-C₃alkyl, or C₁-C₃haloalkyl, or
where the two R^{x} groups, the two R^{y} groups, one R^{x} and one R^{y} group, or one R^{y} and R^{z} group form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
G³ is —OH, —OR^{a}, —SR^{a}, —N(R^{c})₂, heterocycloalkyl, aryl, heteroaryl, or a carbohydrate group linked via an O-, N- or S-glycosidic bond;
wherein each occurrence of R^{c} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{c} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{c} form a 5- or 6-membered cyclic group having 0, 1, or 2 additional heteroatoms chosen from N, O, or S;
G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁵ is —OH, —OR^{a}, —SR^{a}, —N(R^{c})₂, heterocycloalkyl, aryl, heteroaryl, or a carbohydrate group linked via an O-, N- or S-glycosidic bond, and Wand R^{c} are as previously defined;
G⁶ is H, —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
or where the G³ and G⁴ groups or G⁴ and G⁵ groups or G³ and G⁶ groups form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
with the proviso that for D)
1) when G⁶ is H or OMe, G⁵ is OMe, X¹ is O, G¹ is OH, OMe or SMe, both R^{x} is H, both R^{y} is H, and R^{z} is H, then G³ and G⁴ do not form a 5-membered ring containing the group —OCH₂O— or —CH₂CH₂ CH_{z}—;
2) when G⁶ is H, G⁵ is OMe, X¹ is O, G¹ is OMe, both R^{x} is H, both R^{y} is H, and R^{z} is H, then G³ and G⁴ do not form a 6-membered ring containing the group —CH═CH—CH═CH—;
3) when G⁶ is H, G⁵ is OMe, X¹ is O, G¹ is SMe, both R^{x} is H, both R^{x} is H, and R^{z} is H, then G³ and G⁴ do not form a fused 6-membered ring containing two nitrogen atoms;
4) when G⁶ is H, G³ is OMe, X¹ is O, G¹ is SMe, both R^{x} is H, both R^{y} is H, and R^{z} is H, then G⁴ and G⁵ do not form a fused 6-membered ring containing two nitrogen atoms;
5) when G⁵ is OMe, G⁴ is OMe, X¹ is O, G¹ is SMe, both R^{x} is H, both R^{y} is H, and R^{z} is H, then G³ and G⁶ do not form a 5-membered ring containing the group —O—CH═N—; or
6) when G⁶ is H, G³ is OMe, X¹ is O, G¹ is OH or OMe, both R^{x} is H, both R^{y} is H, and R^{z} is H, then G⁴ and G⁵ do not form a 5-membered ring containing the group —OCH₂O—;
or a pharmaceutically acceptable salt, solvate, hydrate, crystalline form, non-crystalline form, or stereoisomer thereof.

2. The compound of claim 1, comprising the S isomer at *.

3. The compound of claim 1, wherein
A) when G¹ is SeR¹, TeR¹, PoR¹, P(R²)₂, N(R³)₂, or Si(R⁴)₃;
R¹ is hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl, wherein when R¹ is not hydrogen it is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R² is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl; or the two R² form a 5- or 6-membered cyclic group having 0, 1, or 2 additional heteroatoms chosen from N, O, or S; wherein each occurrence of R² that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R³ is independently chosen from C₈-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R³ form a 5- or 6-membered cyclic group having 0, 1, or 2 additional heteroatoms chosen from N, O, or S; wherein each occurrence of R³ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
wherein each occurrence of R⁴ independently is hydrogen, C₁-C₁₀ alkyl or C₃-C₇ cycloalkyl; wherein each occurrence of R⁴ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
X¹ is O or S;
G² is —OH, —OR^{a}, —SR^{a}, —N(R^{b})₂, heterocycloalkyl, aryl, heteroaryl, or a carbohydrate group linked via an O-, N- or S-glycosidic bond;
wherein R^{a} is C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, epoxide, carbonate, or N-succinimidyl group; wherein R^{a} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
wherein each occurrence of R^{b} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{b} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{b} form a 5- or 6-membered cyclic group having 0, 1, or 2 additional heteroatoms chosen from N, O, or S;
each occurrence of R^{x}, R^{y}, and R^{z} is independently chosen from hydrogen, halogen, C₁-C₃alkyl, or C₁-C₃haloalkyl;
G³ is —OH, —OR^{a}, —SR^{a}, —N(R^{c})₂, heterocycloalkyl, aryl, heteroaryl, or a carbohydrate group linked via an O-, N- or S-glycosidic bond;
wherein each occurrence of R^{c} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{c} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{c} form a 5- or 6-membered cyclic group having 0, 1, or 2 additional heteroatoms chosen from N, O, or S;
G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁵ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
or where the G³ and G⁴ groups or G⁴ and G⁵ groups form a cyclic 5- or 6-membered ring having two heteroatoms selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
G⁶ is hydrogen;
or a pharmaceutically acceptable salt, solvate, hydrate, crystalline form, non-crystalline form, or stereoisomer thereof.

4. The compound of claim 3, wherein G³ is —OH, G⁴ is —OMe, G⁵ is —OMe, G⁶ is hydrogen, R^{x} is hydrogen, R^{y} is hydrogen, R^{z} is hydrogen, G² is —N(H)Ac, and X¹ is O.

5. The compound of claim 1, wherein
B) when at least one of G², G³, G⁴, or G⁵ is —OR^{a}, —SR^{a}, —N(R^{b})₂, or —N(R^{c})₂ wherein a R^{a}, a R^{b} or a R^{c} is -L-PEG, then the remaining G², G³, G⁴, and G⁵ are as previously defined;
G¹ is OR¹, SR¹, SeR¹, TeR¹, PoR¹, P(R²)₂, N(R⁵)₂, or Si(R⁴)₃, where R¹, R² and R⁴ are as previously defined;
wherein each occurrence of R⁵ is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C_{z}—C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R⁵ form a 5- or 6-membered cyclic group having 0, 1, or 2 additional heteroatoms chosen from N, O, or S; wherein each occurrence of R⁵ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
X¹ is O or S; and
R^{x}, R^{y}, and R^{z} are as previously defined;
or a pharmaceutically acceptable salt, solvate, hydrate, crystalline form, non-crystalline form, or stereoisomer thereof.

6. The compound of claim 5, wherein G³ is -L-PEG, G⁴ is —OMe, G⁵ is —OMe, G⁶ is hydrogen, R^{x} is hydrogen, R^{y} is hydrogen, R^{z} is hydrogen, G² is —N(H)Ac, X¹ is O, and G¹ is —SMe.

7. The compound of claim 1, wherein
C) when at least one of R^{x}, R^{y}, and R^{z} is not hydrogen, then the remaining R^{x}, R^{y}, and R^{z} are as previously defined;
G¹ is OR¹, SR¹, SeR¹, TeR¹, PoR¹, P(R²)₂, N(R⁵)₂, or Si(R⁴)₃, where R¹, R², R⁴ and R⁵ are as previously defined;
X¹ is O or S;
G² is —OH, —OR^{a}, —SR^{a}, —N(R^{b})₂, heterocycloalkyl, aryl, heteroaryl, or a carbohydrate group linked via an O-, N- or S-glycosidic bond, wherein R^{a} and R^{b} are as previously defined;
G³ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined; and
G⁵ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
or a pharmaceutically acceptable salt, solvate, hydrate, crystalline form, non-crystalline form, or stereoisomer thereof.

8. The compound of claim 7, wherein G³ is —OH, G⁴ is —OMe, G⁵ is —OMe, G⁶ is hydrogen, R^{x} is hydrogen, R^{y} is hydrogen, R^{z} is Me, G² is —N(H)Ac, X¹ is O, and G¹ is —SMe.

9. The compound of claim 1, wherein
D) wherein
G¹ is OR¹ or SR¹;
R¹ is hydrogen, C₁-C₁₀ alkyl or C₁-C₁₀ haloalkyl;
X¹ is O;
G² is —OR^{a}, —SR^{a}, or —N(R^{b})₂;
wherein R^{a} is C₁-C₁₀ alkyl, C₂-C₁₀ alkenyl, C₃-C₇ cycloalkyl, C₁-C₁₀ haloalkyl, heterocycloalkyl, aryl, or heteroaryl;
wherein each occurrence of R^{b} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, or C₂-C₆ alkanoyl;
each occurrence of R^{x}, R^{y}, and R^{z} is independently chosen from hydrogen, halogen, C₁-C₃alkyl, or C₁-C₃haloalkyl, or
where the two R^{x} groups, the two R^{y} groups, one R^{x} and one R^{y} group, or one R^{y} and R^{z} group form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy.

10. A compound according to structure (VII), (VIII), or (IX) 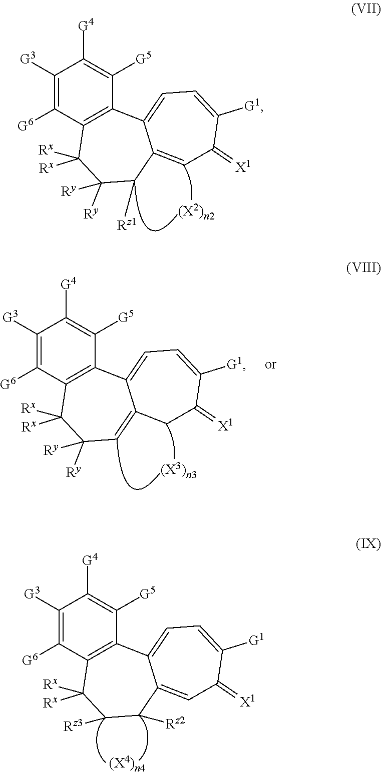
wherein G¹ is OR¹, SR¹, SeR¹, TeR¹, PoR¹, P(R²)₂, N(R⁵)₂, or Si(R⁴)₃, where
R¹ is hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl, wherein when R¹ is not hydrogen it is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R² is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, or heteroaryl; or the two R² form a 5- or 6-membered cyclic group having 0, 1, or 2 additional heteroatoms chosen from N, O, or S; wherein each occurrence of R² that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R⁴ independently is hydrogen, C₁-C₁₀ alkyl, C₂-C₁₀ alkenyl, or C₃-C₇ cycloalkyl; wherein each occurrence of R⁴ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R⁵ is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; or the two R⁵ form a 5- or 6-membered cyclic group having 0, 1, or 2 additional heteroatoms chosen from N, O, or S; wherein each occurrence of R⁵ that is not hydrogen is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
X¹ is O or S;
G³ is —OH, —OR^{a}, —SR^{a}, —N(R^{c})₂, heterocycloalkyl, aryl, heteroaryl, or a carbohydrate group linked via an O-, N- or S-glycosidic bond;
wherein each occurrence of R^{c} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{c} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{c} form a 5- or 6-membered cyclic group having 0, 1, or 2 additional heteroatoms chosen from N, O, or S;
G⁴ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁵ is —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
G⁶ is H, —OH, —OR^{a}, —SR^{a}, —N(R^{c})₂, heterocycloalkyl, aryl, heteroaryl, or a carbohydrate group linked via an O-, N- or S-glycosidic bond, and R^{a} and R^{c} are as previously defined;
or where the G³ and G⁴ groups or G⁴ and G⁵ groups or G³ and G⁶ groups form a cyclic 5- or 6-membered ring optionally having one or two heteroatoms independently selected from O, S, or N, wherein the 5- or 6-membered ring is optionally substituted with 1 or 2 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy;
each occurrence of R^{x}, and R^{y} is independently chosen from hydrogen, halogen, C₁-C₃alkyl, or C₁-C₃haloalkyl
each occurrence of X² is independently O, N, NR^{b}, CR^{b}, or CR^{b}₂ to form a cyclic group, where R^{b} is wherein each occurrence of R^{b} is independently chosen from hydrogen, C₁-C₁₀ alkyl, C₂-C₁₀ alkenyl, C₃-C₇ cycloalkyl, C₁-C₁₀ haloalkyl, heterocycloalkyl, aryl, heteroaryl, C₁-C₆ alkoxycarbonyl, C₂-C₆ alkanoyl, or -L-PEG wherein L is a linking group and PEG is a polyethylene glycol group having a number average molecular weight of about 400 to about 5000, the linking group may be a group derived from an isocyanate, carbonate, or N-succinimidyl group; wherein each occurrence of R^{b} is optionally substituted with 1, 2, or 3 substituents independently chosen from hydroxy, amino, cyano, halogen, C₁-C₆alkyl, C₂-C₆ alkenyl, C₃-C₇cycloalkyl, C₁-C₆alkoxy, mono- and di-(C₁-C₄alkyl)amino, —COOH, C₂-C₆alkoxycarbonyl, C₁-C₆haloalkyl, or C₁-C₆haloalkoxy; or the two R^{b} form a 5- or 6-membered cyclic group having 0, 1, or 2 additional heteroatoms chosen from N, O, or S;
n² is 2 or 3;
R^{z1} is hydrogen or a bond forming the ring structure containing X²;
each occurrence of X³ is O, N, NR^{b}, CR^{b}, or CR^{b}₂ to form a cyclic group where R^{b} is as previously defined;
n³ is 2 or 3;
each instance of X⁴ is O, N, NR^{b}C, CR^{b}, or CR^{b}₂, to form a saturated or unsaturated cyclic group optionally fused to a 5- or 6-membered cyclic or aryl group where R^{b} is as previously defined;
n⁴ is 3 or 4;
R^{z2} is hydrogen or a bond forming the ring structure containing X⁴; and
R^{z3} is hydrogen, hydroxyl, or a bond forming the ring structure containing X⁴;
with the proviso that
a. for structure (VII)
1) when G⁶ is H, G³ is OMe, G⁴ is OMe, G⁵ is OMe, X¹ is O, G¹ is SMe, both R^{x} are H, and both R^{y} are H, then n² is not 2, R^{z1} is not a bond, and X² is not part of a 5-membered ring containing the group ═N—O— (where the oxygen is attached to the tropone ring), or
2) when G⁶ is H, G³ is OMe, G⁴ is OMe, G⁵ is OMe, X¹ is O, G¹ is OMe, both R^{x} is H, and both R^{y} is H, then n² is not 2, R^{z1} is not H, and X² is not part of a 5-membered ring containing the group —N(Ac)—C(OH)(Me)— (where the carbon is attached to the tropone ring);
b. for structure (VIII)
when G⁶ is H, G³ is OMe, G⁴ is OMe, G⁵ is OMe, X¹ is O, G¹ is OMe, both R^{x} is H, and both R^{y} is H, then n³ is not 2 and X³ is not part of a 5-membered ring containing the group —NH—C(CHO)═ or —NH—C(Me)═ where the doublebond is bonded to the tropone ring; or
c. for structure (IX)
1) when G⁶ is H, G³ is OMe, G⁴ is OMe, G⁵ is OMe, X¹ is O, G¹ is OMe or SMe, and both R^{x} is H, then n⁴ is not 4 and X⁴ is not part of a fused 6-membered ring containing the group 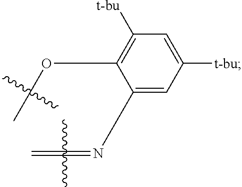
or
2) when G⁶ is H, G³ is OMe, G⁴ is OMe, G⁵ is OMe, X¹ is O, G¹ is OMe, and both R^{x} is H, then n⁴ is not 3 and X⁴ is not part of a 5-membered ring containing the group ═N—N(Ac)—CH═,
or a pharmaceutically acceptable salt, solvate, hydrate, crystalline form, non-crystalline form, or stereoisomer thereof.

11. A pharmaceutical composition, comprising the compound of claim 1 and a pharmaceutically acceptable excipient.

12. The composition of claim 11, formulated for oral, buccal, sublingual, mucosal, transdermal, rectal, vaginal, subcutaneous, intramuscular, or intravenous delivery.

13. The composition of claim 11, formulated as an orally disintegrating tablet or an orally dissolving strip.

14. A method of treating a patient in need of a muscle relaxant, anti-inflammatory agent, an anti-gout agent, an anti-proliferative agent, or an anti-cancer agent comprising administering to the patient the compound of claim 1.

15. A method of treating a patient in need of a muscle relaxant, anti-inflammatory agent, an anti-gout agent, an anti-proliferative agent, or an anti-cancer agent, comprising administering to the patient the composition of claim 11.
